(19) United States
(12) Patent Application Publication
(10) Pub. No.: US 2023/0364445 A1
(43) Pub. Date: Nov. 16, 2023

(54) Method for operating a medical radiation therapy arrangement, and medical radiation therapy arrangement

(71) Applicant: Carl Zeiss Meditec AG, Jena (DE)

(72) Inventor: Gerald Panitz, Bopfingen (DE)

(21) Appl. No.: 18/317,996

(22) Filed: May 16, 2023

(30) Foreign Application Priority Data
May 16, 2022 (DE) 10 2022 204 792.3

(51) Int. Cl. A61N 5/10

(52) U.S. Cl. CPC A61N 5/1039; A61N 5/1067; A61N 5/1083

(57) ABSTRACT

A method for operating a medical radiation therapy arrangement is disclosed, wherein a wound cavity in the patient from which a tumor has been surgically removed is measured three-dimensionally in a reference coordinate system via a surgical microscope of the medical radiation therapy arrangement and/or via a probe-based registration device of the medical radiation therapy arrangement, wherein an x-ray applicator of an intraoperative radiation therapy device of the medical radiation therapy arrangement is selected and/or arranged in the wound cavity on the basis of three-dimensional measurement data generated during the measurement, with the x-ray applicator being navigable in the reference coordinate system. Furthermore, the disclosure relates to a medical radiation therapy arrangement.

### CROSS-REFERENCE TO RELATED APPLICATIONS

This application claims priority of German patent application no. 10 2022 204 792.3, filed May 16, 2022, the entire content of which is incorporated herein by reference.

### TECHNICAL FIELD

The disclosure relates to a method for operating a medical radiation therapy arrangement, and to a medical radiation therapy arrangement.

### BACKGROUND

During cancer therapy, it may be desirable following the removal of a tumor to irradiate the region in which the tumor was located as part of intraoperative radiation therapy. In this way, tumor cells that remain can be destroyed.

U.S. Pat. No. 9,557,158 discloses a system for ascertaining the position of objects in an irradiation room for radiation therapy, including a plurality of room lasers which are arranged in the irradiation room and are each embodied to project at least one laser line onto the surface of a patient situated on a patient table in the irradiation room for the purpose of positioning that patient, further including at least one camera embodied to detect at least one laser line projected by at least one of the room lasers onto the surface of the patient, and including an evaluation and control device, which is embodied to ascertain the coordinate points along the laser line projected onto the surface of the patient during an irradiation procedure on the basis of the measurement values detected by the camera using a real-time triangulation method and to compare them to target coordinate points, wherein the control and evaluation device furthermore includes a memory device in which the coordinate points ascertained during the irradiation procedure are stored for documenting the irradiation procedure. Furthermore, a corresponding method is also described.

### SUMMARY

It is an object of the disclosure to improve a method for operating a medical radiation therapy arrangement and improving a medical radiation therapy arrangement, in particular for use during intraoperative radiation therapy.

According to the disclosure, the above object is, for example, achieved by a method for operating a medical radiation therapy arrangement. The method includes:
three-dimensionally measuring, in a reference coordinate system, a wound cavity in a patient from which a tumor has been surgically removed, wherein said three-dimensional measuring is performed via at least one of a surgical microscope of the medical radiation therapy arrangement and a probe-based registration device of the medical therapy arrangement; and,
at least one of selecting and arranging an x-ray applicator of an intraoperative radiation therapy device of the medical radiation therapy arrangement in the wound cavity on a basis of three-dimensional measurement data generated during said three-dimensional measuring with the x-ray applicator being navigable in the reference coordinate system.

The above mentioned object is, for example, also achieved by a medical radiation therapy arrangement which includes:
at least one of a surgical microscope and a probe-based registration device;
said at least one of said surgical microscope and said probe-based registration device being configured to three-dimensionally measure in a reference coordinate system a wound cavity in a patient from which a tumor was surgically removed;
an intraoperative radiation therapy device having an x-ray applicator;
said x-ray applicator being navigable in the reference coordinate system; and,
said intraoperative radiation therapy device being configured to at least one of select the x-ray applicator and arrange the x-ray applicator in the wound cavity on a basis of three-dimensional measurement data generated during the measurement.

One of the core ideas of the disclosure is to three-dimensionally measure in a reference coordinate system a wound cavity left behind after a tumor is removed (resection). In particular, both the wound cavity and also an entrance and/or a canal to the wound cavity are three-dimensionally measured in the process. In this case, the wound cavity and/or the entrance and/or the canal to the wound cavity are measured in particular topographically. In particular, a surface of the wound cavity is three-dimensionally measured here. The measurement can be effected via a surgical microscope of the medical radiation therapy arrangement, for example by stereoscopically capturing and determining a three-dimensional topology of the wound cavity, of the entrance and/or of the canal to the wound cavity. For example, methods which are known per se, such as triangulation, can be used in this case in order to determine, in the case of known poses and imaging properties of cameras of a stereoscopic camera of the surgical microscope, positions of mutually corresponding image elements in captured stereoscopic image representations in the reference system. Due to the geometry of the wound cavity and/or of the entrance and/or of the canal, capturing from different directions and/or distances may be necessary here.

Alternatively or additionally, the measurement can also be performed via a probe-based registration device of the medical radiation therapy arrangement. Here, the wound cavity, the entrance and/or the canal are measured point by point via a registration device. Contact-based probing and/or probing based on light markings can be provided here. In the case of contact-based probing, the wound cavity, the entrance and/or the canal are probed or brought into contact point by point via a probe head. A pose (position and orientation) of the probe head in the reference coordinate system is known in this case. The pose is captured and/or determined for example via a surgical navigation system in a manner known per se. If probing is carried out on the basis of light markers, light markings are generated via a light source on a surface of the wound cavity and/or the entrance and/or the canal. Due to the geometry of the wound cavity and/or of the entrance and/or of the canal, capturing from different directions and/or distances may be necessary here. The point-type light markings generated are captured via a stereoscopic camera system, for example a stereoscopic camera system of the surgical microscope or of a surgical navigation system, and the position thereof in the reference coordinate system is determined via triangulation in a manner known per se.

The measurement provides, as the result, three-dimensional measurement data for the wound cavity and/or the entrance and/or the canal.

A further core idea of the disclosure is the use of three-dimensional measurement data generated during the measurement to select an x-ray applicator of an intraoperative radiation therapy device of the medical radiation therapy arrangement, and/or arrange it in the wound cavity, on the basis of the data, with the x-ray applicator being navigable in the reference coordinate system. In particular, provision is made for the three-dimensional measurement data to be transmitted as raw data and/or in an already processed form to the intraoperative radiation therapy device via hardware and/or software interfaces that are configured herefor. The selecting can include for example selecting from a magazine in which a multiplicity of x-ray applicators of different sizes and different shapes are held (in particular in sterile fashion) and from which the x-ray applicators can be removed as required. In principle, the x-ray applicator can be arranged in this case both manually and in automated, for example partially or fully automated, fashion, wherein the basis for the arrangement in both cases are the three-dimensional measurement data of the wound cavity, of the entrance and/or of the canal.

In particular, a method for operating a medical radiation therapy arrangement is provided, wherein a wound cavity in the patient from which a tumor has been surgically removed is measured three-dimensionally in a reference coordinate system via a surgical microscope of the medical radiation therapy arrangement and/or via a probe-based registration device of the medical radiation therapy arrangement, wherein an x-ray applicator of an intraoperative radiation therapy device of the medical radiation therapy arrangement is selected and/or arranged in the wound cavity on the basis of three-dimensional measurement data generated during the measurement, with the x-ray applicator being navigable in the reference coordinate system.

Furthermore, in particular a medical radiation therapy arrangement is created, including a surgical microscope and/or a probe-based registration device, wherein these are configured to three-dimensionally measure in a reference coordinate system a wound cavity in the patient from which a tumor has been surgically removed; and an intraoperative radiation therapy device having an x-ray applicator which is navigable in the reference coordinate system, wherein the intraoperative radiation therapy device is configured to select the x-ray applicator and/or arrange it in the wound cavity on the basis of three-dimensional measurement data generated during the measurement.

One advantage of the method and of the medical radiation therapy arrangement is that irradiation of the wound cavity following the resection of the tumor can be carried out with greater positional accuracy. In particular, there is a direct exchange of the three-dimensional measurement data between the surgical microscope and/or the registration device and the intraoperative radiation therapy device. For this purpose, the surgical microscope and/or the registration device and the intraoperative radiation therapy device have in particular suitable interfaces for data exchange. Another advantage is that a suitable x-ray applicator can be selected in a targeted manner on the basis of the three-dimensional measurement data captured, rather than on the basis of human estimation or trial and error. Furthermore, the safety during the insertion of the applicator with an alignment that is an exact fit with the wound canal can be improved.

In particular, provision is made for poses (positions and orientations) of the surgical microscope and/or of the registration device and of the x-ray applicator in the reference system to be known. The poses can be determined with the aid of methods that are known per se, for example via an additional navigation system or using suitable marks that are captured and evaluated by the surgical microscope. In principle, other methods and/or sensor systems can also be used to determine the poses.

Provision may be made for the surgical microscope and/or the registration device and the intraoperative radiation therapy device to each include control devices and/or data processing devices. Provision may be made in this case for at least one of the control devices and/or the data processing devices to process and/or evaluate the three-dimensional measurement data. Processing can include, for example, combining and/or smoothing and/or averaging measurement points captured.

Provision may also be made for the surgical microscope and/or the registration device and the intraoperative radiation therapy device to not be directly interconnected, but for the three-dimensional measurement data to be exchanged via a (central) data processing device to which the surgical microscope and/or the registration device and the intraoperative radiation therapy device are connected. Provision may be made in this case for the data processing device to process and/or evaluate the three-dimensional measurement data. Processing can include, for example, combining and/or smoothing and/or averaging measurement points captured. The (central) data processing device can also provide one or both of the control devices of the surgical microscope and/or of the intraoperative radiation therapy device.

Provision may be made, for example, for a size and/or a volume and/or a shape of the wound cavity and/or of the entrance and/or of the canal to the wound cavity to be determined in order to select the x-ray applicator. On that basis it is then possible to select a suitable applicator from a multiplicity of x-ray applicators of different sizes and/or shapes that are held available. This can take place in particular in automated fashion from a magazine. Provision may be made in this case for a site of the tumor before removal to be additionally determined from pre-operatively captured measurement data and for regions of increased risk of tumor tissue being left behind to be determined based on the determined size and shape of the wound cavity. Furthermore, fluorescence imaging with the surgical microscope can also identify such regions. Similar to fluorescence imaging or other tissue-diagnosis methods and/or apparatuses integrated in the microscope, confocal endomicroscopy or other (probe-based) methods can be used to identify tumor tissue left behind, as will be described further below. If the probes of these methods and/or apparatuses are navigated, the site of the tissue in the wound cavity can be contained in the three-dimensional measurement data for the radiation therapy device.

The arrangement takes place in particular after the three-dimensional measurement data have been evaluated. For this purpose, for example a position and/or orientation to be adopted during irradiation by the selected x-ray applicator can be determined. If the x-ray applicator for example has a spherical shape in the active region, the position can be determined as the center of the sphere. The position provided for the arrangement can be determined for example by determining a center of the wound cavity from the three-dimensional measurement data. The center of the sphere is then arranged at the determined position for the purposes of irradiation. The orientation is determined in particular on the basis of information about a course of the entrance and/or of the canal to the wound cavity contained in the three-dimensional measurement data because the x-ray applicator is inserted via the entrance and/or the canal into the wound cavity and held in position there. It should be noted that a position of the patient during the performance of the method in particular remains constant or can be determined again in the case of repositioning in the reference coordinate system, for example using optical marks arranged fixedly on the patient so that the three-dimensional measurement data can be correspondingly transformed.

A surgical microscope can be in particular a KINEVO-type microscope from Carl Zeiss Meditec AG. An intraoperative radiation therapy device (IORT) can in particular be an INTRABEAM-type device from Carl Zeiss Meditec AG. As part of this disclosure, a confocal endomicroscope (CEM) may be in particular of the type CONVIVO from Carl Zeiss Meditec AG.

Provision is made in an embodiment, for arrangement purposes, for a feed trajectory for the x-ray applicator to and/or into the wound cavity to be determined and/or output and/or implemented by moving the x-ray applicator, on the basis of the generated three-dimensional measurement data. It is possible to take into account here in particular that the wound cavity is generally not accessible from all directions but from only one direction that coincides with the entrance and/or the canal to the wound cavity. The trajectory is then determined such that the x-ray applicator is inserted into the wound cavity from the direction of the entrance and/or along the canal. For this purpose, the trajectory in particular also includes for each position information relating to the orientation (or alignment) of the x-ray applicator. In a simple case, it is possible for the entrance and/or the canal to be approximated as a cylinder or to be adapted or fitted to the three-dimensional measurement data. The feed trajectory is then determined such that the position thereof extends along an axis running along the middle of the cylinder, wherein the feed trajectory terminates for example at the center of the wound cavity. In a simple case, after the removal of the tumor the wound cavity consists for example of a sphere or hemisphere (in accordance with the tumor, which frequently exhibits spherical growth) and a cylinder along an entrance that is created by the surgeon and adapted to anatomical conditions. The surgical microscope can determine a shape and a site of the entrance in the known reference coordinate system either as the surgery progresses or in a separate maneuver at the end of (or after) the tumor removal. If a trocar (cylindrical or slightly conical metal tube) has been inserted, the entrance (also referred to as wound canal) is geometrically identical to a central axis of the trocar. If the site of the wound cavity is slightly asymmetric, the ideal canal is a straight connection between a wound cavity center and a center of the canal beginning. For this purpose, the surgical microscope is aligned in particular with a wound cavity center of the wound cavity (in particular the image center or the focal point of the surgical microscope). Next, the user, possibly supported by the surgical microscope, would pivot the latter around the wound cavity center (that is, move on a universal ball joint at a fixed radius) or the surgical microscope would itself pivot robotically until the wound canal as a whole or at least the entrance opening is arranged centrally around a viewing axis of the surgical microscope. Next, a site of an image axis of the surgical microscope is determined in the reference coordinate system, and at least the bottom of the wound cavity is ascertained as its deepest extent.

Provision is made in an embodiment for the arrangement of the x-ray applicator to be performed via a robotic stand on which the x-ray applicator is arranged. In this way, an automated arrangement of the x-ray applicator in the wound cavity can be performed. Provision may be made here for only a first part of a feed trajectory to be implemented in automated fashion, while a second part at the end of the feed trajectory is implemented manually, wherein a surgeon or an assistant controls the robotic stand for inserting the x-ray applicator. Provision may be made in the second part for the surgeon or the assistant to be provided here with information about deviating from an ideal movement path on the feed trajectory, for example by acoustic and/or visual signaling and/or by increased virtual resistance of the robotic stand.

Provision is made in an embodiment as part of selecting the x-ray applicator for at least one external part of the x-ray applicator to be produced taking into account the three-dimensional measurement data. In this way, at least the outer part of the x-ray applicator can be adapted to an actual three-dimensional topographical course of the wound cavity (that is, a course determined on the basis of the actual shape of the wound cavity that is present after the tumor has been removed, rather than pre-operatively determined). An x-ray applicator of this type that is produced in a customized manner can be inserted with accurate fit into the wound cavity so that controlled irradiation can take place. It is also possible hereby to prevent displacement of tissue during the irradiation because the tissue can be held in position on account of the “negative shape” of the x-ray applicator. In principle, the outer part of the x-ray applicator can, however, also have a different shape produced taking into account the three-dimensional measurement data. Provision can here also be made for a search for a (standard) x-ray applicator that is present (for example from a magazine held available) to be carried out on the basis of the three-dimensional measurement data generated during the measurement. If no such (standard) x-ray applicator is found, at least the outer part of the x-ray applicator is produced taking into account the three-dimensional measurement data.

In an embodiment, provision is made for the wound cavity to be measured additionally via a confocal endomicroscope of the medical radiation therapy arrangement and/or via another apparatus for tissue differentiation, in particular another apparatus of the medical radiation therapy arrangement, wherein a density of tumor cells left behind in the wound cavity after the surgical removal of the tumor and/or another tissue characteristic that is relevant for the therapy is determined in the reference coordinate system in a spatially resolved manner, and wherein the selection of the x-ray applicator takes place taking into account the density of the tumor cells determined in a spatially resolved manner and/or the other tissue characteristic that is relevant for the therapy and/or wherein at least an outer part of the x-ray applicator is produced taking into account the density of the tumor cells determined in a spatially resolved manner and/or the other tissue characteristic that is relevant for the therapy. In this way, different regions of the wound cavity can be irradiated with a different irradiance (intensity or dose), that is, inhomogeneously with respect to a surface area or anisotropically, in dependence on the density of the remaining tumor cells and/or of the other tissue characteristic that is relevant for the therapy. For example, regions in which a greater density of remaining tumor cells has been determined can be irradiated more strongly than regions in which the density of the tumor cells is lower. This also allows the protection of regions in which tumor cells are no longer present, or which should not be irradiated or should be irradiated less strongly for other reasons, against excess irradiation. In particular a wall thickness (that is, a thickness) and/or a material of a shielding material of the applicator and/or a shape can be taken into account here when selecting the x-ray applicator. Different wall thicknesses lead to a more or less pronounced attenuation of the x-rays. A drop in intensity of the x-rays indicates an exponential dependence on the wall thickness. Likewise, an attenuation can occur owing to the selection of the respectively used material. The values for the wall thickness and/or the material properties can be in particular a spatially dependent function of the determined density of the tumor cells. Furthermore, a structure of the x-ray applicator can also be selected differently in order to vary an irradiance in dependence on the determined density. For example, cavities can reduce an effective wall thickness (for example a honeycomb structure can be selected). Another apparatus for tissue differentiation can be, for example, an optical coherence tomography (OCT) apparatus or a Raman spectroscopy apparatus. Furthermore, provision may be made for the other apparatus to be provided by the surgical microscope in the form of fluorescence imaging.

In an embodiment, provision is made for at least the outer part of the x-ray applicator to be produced via a 3D printing method. Control data and/or structure data required for this purpose can be generated and provided for example via one of the control devices and/or the (central) data processing device. In particular, provision may be made for the x-ray applicator to be produced via a 3D printing method taking into account the density of the tumor cells determined in a spatially resolved manner. Here, a wall thickness (material thickness) and/or a material and/or a structure of the x-ray applicator are selected in a spatially resolved manner in particular such that, during subsequent irradiation, a respectively desired irradiance (intensity or dose) is attained in a spatially resolved manner. The 3D printing method is in particular a sterile 3D printing method.

In an embodiment, provision is made for marked regions adjoining the tumor and/or the wound cavity to be captured and/or identified, and/or for measurement and/or simulation data describing these marked regions to be received, wherein these data are taken into account during the selection and/or during the production of the x-ray applicator. It is hereby possible, especially during brain surgery, for functionally active areas and/or bundles of nervous fibers to be protected in a targeted manner during the irradiation.

In an embodiment, provision is made for three-dimensional external measurement data describing the tumor that is to be removed to be received, wherein a start position and/or a start orientation at least for the surgical microscope and/or the intraoperative radiation therapy device are determined in the reference coordinate system on the basis of the received three-dimensional external measurement data, and wherein the surgical microscope and/or the intraoperative radiation therapy device are brought into the respectively determined start position and/or the respectively determined start orientation in the reference coordinate system. In this way, the surgical microscope and/or the intraoperative radiation therapy device can be brought into a suitable position and orientation (or alignment) even before a resection or before the irradiation. The external measurement data can be captured for example via magnetic resonance imaging (MRI) or via a different suitable method. During preoperative imaging, the body of the patient is imaged at least in such a region and in a manner such that uniquely identifiable features, such as for example the tip of the nose, nasal root, eyebrow ridges and cheekbones et cetera, are contained in the dataset of the external measurement data. During the operation, the patient is measured by the navigation system, that is, a site of the unique features is determined in a fixed coordinate system, in particular in the reference coordinate system. In the further process, all other surgical devices, such as the surgical microscope, the endoscope, and the intraoperative radiation therapy device, are typically likewise registered with the reference coordinate system on the basis of marks arranged on the respective device or on a probe of the respective device that is relevant for navigation. For example, if the surgeon now defines in the pre-operatively captured dataset, that is, in the external measurement data, the center of the cranial opening to be produced on the cranial structure of the patient, this information (exact site of this point with spatial normal, that is, a direction of a perpendicular to the surface at this point) can be transmitted to the surgical microscope. The latter can then align itself such that its viewing axis intersects this point exactly perpendicularly (that is, the viewing axis extends along the spatial normal) and a specific distance from the point is observed. After the cranium is opened, this alignment can be effected for example exactly in the direction of anatomical structures which were determined automatically and/or manually in the preoperatively captured dataset. Analogously, the dataset can be supplemented during the operation by the tissue removed and possibly by the size, shape and alignment of the entrances.

Further features relating to the configuration of the medical radiation therapy arrangement are evident from the description of configurations of the method. Here, the advantages of the medical radiation therapy arrangement are in each case the same as in the configurations of the method.

### BRIEF DESCRIPTION OF DRAWINGS

The invention will now be described with reference to the drawings wherein:

FIG. 1 shows a schematic illustration of an embodiment of the medical radiation therapy arrangement; and,

FIG. 2 shows a schematic flowchart of an embodiment of the method for operating a medical radiation therapy arrangement.

### DETAILED DESCRIPTION

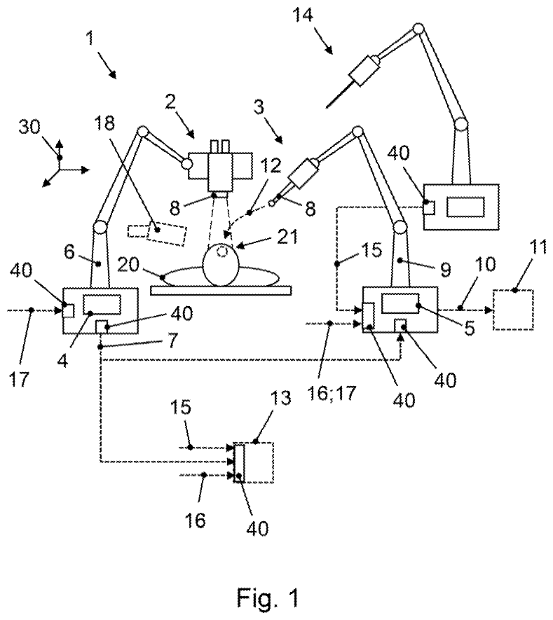

FIG. 1 shows a schematic illustration of an embodiment of the medical radiation therapy arrangement 1. The medical radiation therapy arrangement 1 includes a surgical microscope 2 and an intraoperative radiation therapy device 3. The medical radiation therapy arrangement 1 is configured to carry out the method described in this disclosure. The surgical microscope 2 and the intraoperative radiation therapy device 3 each have a control device 4, 5, which perform data processing that is necessary to carry out measures of the method.

Alternatively, the measures of the method can also be carried out via a common control device, for example a central data processing device.

The individual devices of the radiation therapy arrangement 1, in particular the surgical microscope 2, any central data processing device that may be present, and the intraoperative radiation therapy device 3, are interconnected in particular via suitable hardware and/or software interfaces 40.

The surgical microscope 2 is configured to three-dimensionally measure in a reference coordinate system 30 a wound cavity 21 in a patient 20 from which a tumor was surgically removed. For this purpose, in particular stereoscopic image representations are captured and evaluated via a stereoscopic camera of the surgical microscope 2. A pose of the surgical microscope 2 within the reference coordinate system 30 can be changed for this purpose via a robotic stand 6. In this way, the wound cavity 21 can be captured and measured from different directions. In particular, outer walls of the wound cavity 21 are topographically measured in the process. In particular methods in which three-dimensional positions of the outer wall of the wound cavity 21 are determined via triangulation from the stereoscopic image representations can be used here. The surgical microscope 2 provides three-dimensional measurement data 7 describing the wound cavity 21. The three-dimensional measurement data 7 are supplied to the intraoperative radiation therapy device 3, in particular via the hardware and/or software interfaces 40 that are configured for this purpose.

In particular, provision is made for an entrance and/or a canal to the wound cavity 21 to be three-dimensionally measured in the reference coordinate system 20 in addition to the wound cavity 21.

A pose of the surgical microscope 2 in the reference coordinate system 30 is known, and the positions of the outer wall of the wound cavity 21 can thus be determined from the captured stereoscopic image representations. The pose of the surgical microscope 2 can be determined for example by capturing optical marks via the surgical microscope 2 and/or another sensor system. Alternatively or additionally, a pose of the surgical microscope 2 can also be captured and/or determined via a surgical navigation system (not shown).

Alternatively or additionally, provision may be made for the three-dimensional measurement to be performed via a registration device 18. The registration device 18 is used for example to probe and consequently measure (in particular manually) points within the wound cavity 21. A pose of the registration device 18 in the reference coordinate system 30 is known here, for example by way of capturing optical marks arranged on the registration device 18 via a surgical navigation system. The registration device 18 can alternatively also generate light markings within the wound cavity 18, which are then stereoscopically captured via cameras of the surgical microscope 2 (or of another suitable device), wherein a position of the generated light marking is determined from the stereoscopic image representations captured. In this way, the wound cavity 21 can be captured and measured (in particular manually) step-by-step.

The intraoperative radiation therapy device 3 has an x-ray applicator 8 that is navigable in the reference coordinate system 30. The applicator is arranged in particular at a distal end of a robotic stand 9. In particular, a pose of the x-ray applicator 8 can be changed via the robotic stand 9. The intraoperative radiation therapy device 3 is configured to select the x-ray applicator 8 and/or arrange it in the wound cavity 21 on the basis of the three-dimensional measurement data 7 generated during the measurement. For this purpose, the three-dimensional measurement data 7 are received by the interface 40 of the intraoperative radiation therapy device 3 and processed by the control device 5.

The selection can be carried out for example via a magazine of the radiation therapy device 3 that is held available for this purpose. X-ray applicators 8 of different sizes and/or shapes are stored in sterile fashion in the magazine. Provision is made here in particular for the radiation therapy device 3 to select an x-ray applicator 8 that is suitable for the wound cavity 21 and/or for the entrance and/or the canal to the wound cavity 21. For this purpose, the radiation therapy device 3, in particular the control device 5, determines, for example, a volume of the wound cavity 21 on the basis of the three-dimensional measurement data 7 and selects a suitable x-ray applicator 8 from the applicators 8 that are held available in the magazine, on the basis of the volume determined. The x-ray applicator 8 selected is then in automated fashion taken from the magazine by the radiation therapy device 3 and coupled to the robotic stand 9. Alternatively, the x-ray applicator 8 can also be manually taken from the magazine and connected to the stand 9.

Subsequently, the selected x-ray applicator 8 is arranged in the wound cavity 21. A pose, that is, a position and an orientation, of the x-ray applicator 8, in particular a pose of a part of the x-ray applicator 8 that is active during the irradiation, is determined on the basis of the three-dimensional measurement data 7. In particular, this process is implemented via the control device 5. The arrangement is then performed in particular in automated fashion via the robotic stand 6. In principle, however, a manually guided arrangement is also possible, in which a surgeon or an assistant manually inserts the x-ray applicator 8, supported by the three-dimensional measurement data 7 and the pose determined therefrom, (through the entrance and/or the canal) into the wound cavity 21. For this purpose, for example (three-dimensional) visualization 10 of the three-dimensional measurement data 7 and a pose of the x-ray applicator 8, for example on a display device 11 of the radiation therapy arrangement 1, can take place.

Provision can be made, for arrangement purposes, for a feed trajectory 12 for the x-ray applicator 8 to and/or into the wound cavity 21 to be determined and/or output and/or implemented by moving the x-ray applicator 8, on the basis of the generated three-dimensional measurement data 7. The feed trajectory 12 includes an ordered set of poses that each include a position and an orientation of the x-ray applicator 8 in the reference coordinate system 30. The feed trajectory 12 begins at a current pose of the x-ray applicator 8 and terminates in an end pose in the wound cavity 21. The determination of the feed trajectory 12 is effected in particular via the control device 5. Provision may be made for the determined feed trajectory 12 to be displayed on the display device 11 in order to aid a surgeon or an assistant with the orientation and navigation.

Provision may be made for at least an outer part of the x-ray applicator 8 to be produced taking into account the three-dimensional measurement data 7. For this purpose, the three-dimensional measurement data 7 are supplied for example to a 3D printer 13, which produces an outer part of the x-ray applicator 8 on the basis of the three-dimensional measurement data 7 of the wound cavity 21, of the entrance and/or of the canal. For example, the outer part of the x-ray applicator 8 can be produced as a negative image or negative shape of the wound cavity 21, with the result that the x-ray applicator 8 can be arranged with an exact fit in the wound cavity 21. In principle, it is possible here for a radiologist or an assistant to specify parameters for the production process, for example at a user interface (not shown) that is provided for this purpose. For example, it is also possible to select a region of the wound cavity 21 that is to be produced as a negative shape. The production can take place in a sterile environment. Alternatively or additionally, the produced x-ray applicator 8 can be sterilized after production, for example via methods using UV radiation, ethylene oxide (EtO) and/or a plasma. The produced x-ray applicator 8 is subsequently arranged on the robotic stand 9 and arranged in the wound cavity 21, as already described.

Provision may be made for the medical radiation therapy arrangement 1 to additionally have a confocal endomicroscope 14 and/or another apparatus (not shown) for tissue differentiation. The endoscope or other apparatus are connected to the other devices of the radiation therapy arrangement 1 in particular via a suitable hardware and/or software interface 40. Provision is then made for the wound cavity 21 to be measured additionally via the confocal endomicroscope 14 and/or the other apparatus, wherein a density 15 of tumor cells that are left behind in the wound cavity 21 after the tumor has been surgically removed and/or another tissue characteristic that is relevant for the therapy is determined in a spatially resolved manner in the reference coordinate system 30. The x-ray applicator 8 is then selected taking into account the density 15 of the tumor cells determined in a spatially resolved manner and/or the other tissue characteristic that is relevant for the therapy. Alternatively or additionally, at least the outer part of the x-ray applicator 8 can be produced taking into account the density 15 of the tumor cells determined in a spatially resolved manner and/or the other tissue characteristic that is relevant for the therapy. In particular, the x-ray applicator 8 can here be selected or produced such that regions having a greater density 15 are irradiated with a greater irradiance (intensity or dose) than regions having a lower density 15. Such an x-ray applicator 8 can be produced in particular via the 3D printer 13 by selecting a wall thickness and/or structure (for example a solid configuration or one with cavities, with a honeycomb structure et cetera) of the outer part of the x-ray applicator 8 such that a weakening corresponding thereto results in a desired irradiance (intensity or dose). Alternatively or additionally, a (spatially resolved) material selection (for example by admixing a metal component et cetera) during 3D printing can bring about a desired weakening. Another apparatus for tissue differentiation can be, for example, an optical coherence tomography (OCT) apparatus or a Raman spectroscopy apparatus. Furthermore, provision may be made for the other apparatus to be provided by the surgical microscope in the form of fluorescence imaging.

Provision may be made for marked regions adjoining the tumor and/or the wound cavity 21 to be captured and/or identified, and/or for measurement and/or simulation data 16 describing these marked regions to be received, wherein these data are taken into account during the selection and/or during the production of the x-ray applicator 8. The marked regions can be, for example, functionally active areas and/or bundles of fibers in the brain of the patient 20, which are captured and/or identified using suitable methods.

Provision may be made for three-dimensional external measurement data 17 describing the tumor that is to be removed to be received, wherein a start position and/or a start orientation at least for the surgical microscope 2 and/or the intraoperative radiation therapy device 3 are determined in the reference coordinate system 30 on the basis of the received three-dimensional external measurement data 17, and wherein the surgical microscope 2 and/or the intraoperative radiation therapy device 3 are brought into the respectively determined start position and/or the respectively determined start orientation in the reference coordinate system 30. For this purpose, the control devices 4, 5 determine, on the basis of the three-dimensional external measurement data 17, the respective start positions and/or start orientations in the reference coordinate system 30 and control (in open-loop and/or closed-loop fashion) the robotic stands 6, 9 accordingly.

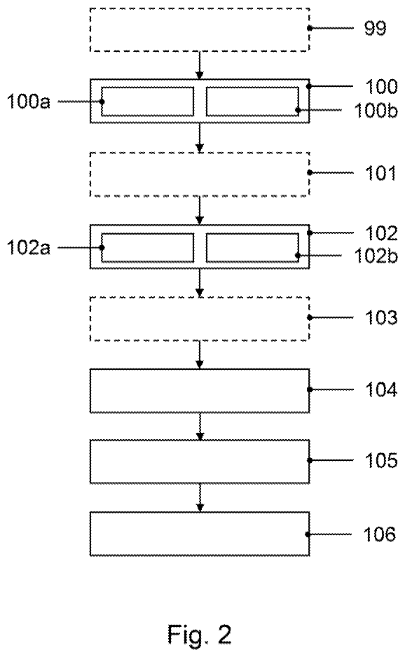

FIG. 2 shows a schematic flowchart of an embodiment of the method for operating a medical radiation therapy arrangement. The method is carried out in particular via a medical radiation therapy arrangement according to any of the previously described embodiments.

In a measure 100, a wound cavity in the patient from which a tumor was surgically removed is three-dimensionally measured in a reference coordinate system. This can be done via a surgical microscope (measure 100a) of the medical radiation therapy arrangement and/or via a probe-based registration device of the medical radiation therapy arrangement (measure 100b). Additionally, in particular a surgical navigation system can be used in this case, with which poses of the surgical microscope and/or of the registration device in the reference coordinate system are determined.

In a measure 102, an x-ray applicator of an intraoperative radiation therapy device of the medical radiation therapy arrangement is selected on the basis of three-dimensional measurement data generated during the measurement (measure 102a), with the x-ray applicator being navigable in the reference coordinate system. Alternatively, provision may also be made, as part of the measure 102, for at least an outer part of the x-ray applicator to be produced taking into account the three-dimensional measurement data (measure 102b). This can be effected in particular via a 3D printing method.

Provision may be made in measure 103 for a feed trajectory for the x-ray applicator to and/or into the wound cavity to be determined and/or output on the basis of the generated three-dimensional measurement data.

In measure 104, the x-ray applicator is arranged in the wound cavity. For this purpose, in particular the feed trajectory determined in measure 103 can be implemented. In a simple case, the arrangement can be effected manually by a surgeon or an assistant. Provision may in particular furthermore be made for the arrangement of the x-ray applicator to be performed via a robotic stand on which the x-ray applicator is arranged. The arrangement via the robotic stand can be effected both under manual control or in automated fashion. Combinations are also possible; for example, provision may be made for the robotic stand to implement the feed trajectory up to a specified position in automated fashion, but for a remaining portion of the feed trajectory to be implemented under manual control by the surgeon or the assistant. For this purpose, provision may be made in particular for the feed trajectory to be displayed on a display device together with a presentation of the three-dimensional measurement data of the wound cavity, of the entrance and/or of the canal to the wound cavity and the current pose of the x-ray applicator.

When the x-ray applicator has been successfully arranged in the wound cavity, the tissue adjoining the wound cavity is irradiated in a measure 105 via the x-ray applicator.

After the irradiation, the x-ray applicator is removed from the wound cavity in a measure 106. This takes place in particular by implementing the feed trajectory in the opposite direction.

Provision may be made in an additional measure 101 for the wound cavity to be measured additionally via a confocal endomicroscope of the medical radiation therapy arrangement and/or via another apparatus for tissue differentiation, wherein a density of tumor cells that are left behind in the wound cavity after the tumor has been surgically removed and/or another tissue characteristic that is relevant for the therapy is determined in the process in a spatially resolved manner in the reference coordinate system. The x-ray applicator is then selected in measure 102a taking into account the density of the tumor cells determined in a spatially resolved manner and/or the other tissue characteristic that is relevant for the therapy. Alternatively, at least an outer part of the x-ray applicator is produced in measure 102b taking into account the density of the tumor cells determined in a spatially resolved manner and/or the other tissue characteristic that is relevant for the therapy.

Provision may furthermore be made in an additional measure 99 for marked regions adjoining the tumor and/or the wound cavity to be captured and/or identified, and/or for measurement and/or simulation data describing these marked regions to be received, wherein these data are taken into account during the selection in measure 102a and/or during the production of the x-ray applicator in measure 102b. Capturing can be effected for example via a magnetic resonance imaging (MRI) method or another suitable imaging method.

Provision may furthermore be made for three-dimensional external measurement data describing the tumor that is to be removed to be received, wherein a start position and/or a start orientation at least for the surgical microscope and/or the intraoperative radiation therapy device are determined in the reference coordinate system on the basis of the received three-dimensional external measurement data, and wherein the surgical microscope and/or the intraoperative radiation therapy device are brought into the respectively determined start position and/or the respectively determined start orientation in the reference coordinate system.

It is understood that the foregoing description is that of the preferred embodiments of the invention and that various changes and modifications may be made thereto without departing from the spirit and scope of the invention as defined in the appended claims.

### LIST OF REFERENCE SIGNS

1 Medical radiation therapy arrangement
2 Surgical microscope
3 Intraoperative radiation therapy device
4 Control device
5 Control device
6 Robotic stand
7 Three-dimensional measurement data
8 X-ray applicator
9 Robotic stand
10 Visualization
11 Display device
12 Feed trajectory
13 3D printer
14 Confocal endomicroscope
15 Density (of tumor cells left behind)
16 Measurement and/or simulation data
17 External measurement data
18 Registration device
20 Patient
21 Wound cavity
30 Reference coordinate system
40 Interface
99-106 Measures of the method

1. A method for operating a medical radiation therapy arrangement, the method comprising:
three-dimensionally measuring, in a reference coordinate system, a wound cavity in a patient from which a tumor has been surgically removed, wherein said three-dimensional measuring is performed via at least one of a surgical microscope of the medical radiation therapy arrangement and a probe-based registration device of the medical therapy arrangement; and,
at least one of selecting and arranging an x-ray applicator of an intraoperative radiation therapy device of the medical radiation therapy arrangement in the wound cavity on a basis of three-dimensional measurement data generated during said three-dimensional measuring with the x-ray applicator being navigable in the reference coordinate system.

2. The method of claim 1 further comprising transmitting the three-dimensional measurement data via interconnected interfaces, which are configured for said transmitting, of at least one of the surgical microscope, the probe-based registration device, a confocal endomicroscope of the medical radiation therapy arrangement, and a further apparatus for tissue differentiation and the intraoperative radiation therapy device.

3. The method of claim 1, wherein, for said arranging, a feed trajectory for the x-ray applicator to and/or into the wound cavity is at least one of determined, output, and implemented by moving the x-ray applicator, on the basis of the generated three-dimensional measurement data.

4. The method of claim 1, wherein said arranging of the x-ray applicator is performed via a robotic stand on which the x-ray applicator is arranged.

5. The method of claim 1, wherein, as part of selecting the x-ray applicator, at least one external part of the x-ray applicator is made taking into account the three-dimensional measurement data.

6. The method of claim 1, wherein the wound cavity is measured additionally via at least one of a confocal endomicroscope of the medical radiation therapy arrangement, and a further apparatus for tissue differentiation, wherein a density of tumor cells left behind in the wound cavity after the surgical removal of at least one of the tumor and an other tissue characteristic that is relevant for the therapy is determined in the reference coordinate system in a spatially resolved manner; and, said selecting of the x-ray applicator takes place taking into account a density of a corresponding one of the tumor cells determined in the spatially resolved manner and the other tissue characteristic that is relevant for the therapy.

7. The method of claim 1, wherein the wound cavity is measured additionally via at least one of a confocal endomicroscope of the medical radiation therapy arrangement, and a further apparatus for tissue differentiation, wherein a density of tumor cells left behind in the wound cavity after the surgical removal of at least one of the tumor and an other tissue characteristic that is relevant for the therapy is determined in the reference coordinate system in a spatially resolved manner; and, at least an outer part of the x-ray applicator is made taking into account the density of a corresponding one of the tumor cells determined in a spatially resolved manner and the other tissue characteristic that is relevant for the therapy.

8. The method of claim 1, wherein the wound cavity is measured additionally via at least one of a confocal endomicroscope of the medical radiation therapy arrangement, and a further apparatus for tissue differentiation, wherein at least one of a density of tumor cells left behind in the wound cavity after the surgical removal of the tumor and an other tissue characteristic relevant for the therapy is determined in the reference coordinate system in a spatially resolved manner; said selecting of the x-ray applicator takes place taking into account the density of a corresponding one of the tumor cells determined in the spatially resolved manner and the other tissue characteristic relevant for the therapy; and, at least an outer part of the x-ray applicator is made taking into account the density of a corresponding one of the tumor cells determined in a spatially resolved manner and the other tissue characteristic that is relevant for the therapy.

9. The method of claim 5, wherein the outer part of the x-ray applicator is made via a 3D printing method.

10. The method of claim 6, wherein the outer part of the x-ray applicator is made via a 3D printing method.

11. The method of claim 1, wherein marked regions adjoin at least one of the tumor and the wound cavity;
wherein at least one of:
the marked regions are captured,
the marked regions are identified,
measurement data describing the marked regions are received, and
simulation data describing the marked regions are received; and,
wherein the data are taken into account during the selection and/or during a making of the x-ray applicator.

12. The method of claim 1, wherein three-dimensional external measurement data describing the tumor that is to be removed are received; at least one of a start position and a start orientation for at least one of the surgical microscope and the intraoperative radiation therapy device are determined in the reference coordinate system on a basis of the received three-dimensional external measurement data; and, at least one of the surgical microscope and the intraoperative radiation therapy device is brought into at least one of a corresponding one of the determined start position and the determined start orientation in the reference coordinate system.

13. A medical radiation therapy arrangement comprising:
at least one of a surgical microscope and a probe-based registration device;
said at least one of said surgical microscope and said probe-based registration device being configured to three-dimensionally measure in a reference coordinate system a wound cavity in a patient from which a tumor was surgically removed;
an intraoperative radiation therapy device having an x-ray applicator;
said x-ray applicator being navigable in the reference coordinate system; and,
said intraoperative radiation therapy device being configured to at least one of select the x-ray applicator and arrange the x-ray applicator in the wound cavity on a basis of three-dimensional measurement data generated during the measurement.

14. The medical radiation therapy arrangement of claim 13, wherein at least one of said surgical microscope, said probe-based registration device, a confocal endomicroscope of the medical radiation therapy arrangement, and a further apparatus for tissue differentiation and said intraoperative radiation therapy device have interconnected interfaces configured to transmit the three-dimensional measurement data.

15. The medical radiation therapy arrangement of claim 13 further comprising:
at least one of a confocal endomicroscope and a further apparatus for tissue differentiation;
said at least one of said confocal endomicroscope and said further apparatus for tissue differentiation being configured to additionally measure the wound cavity, wherein at least one of a density of at least one of tumor cells left behind in the wound cavity after surgical removal of the tumor and another tissue characteristic that is relevant for the therapy is determined in the reference coordinate system in a spatially resolved manner; and,
wherein the selection of said x-ray applicator takes into account at least one of the density of the tumor cells determined in a spatially resolved manner and the other tissue characteristic that is determined.

16. The medical radiation therapy arrangement of claim 15, wherein at least an outer part of the x-ray applicator is produced taking into account at least one of the density of the tumor cells determined in a spatially resolved manner and the other tissue characteristic that is determined.

17. The medical radiation therapy arrangement of claim 13 further comprising:
at least one of a confocal endomicroscope and a further apparatus for tissue differentiation;
said at least one of said confocal endomicroscope and said further apparatus for tissue differentiation being configured to additionally measure the wound cavity, wherein at least one of a density of at least one of tumor cells left behind in the wound cavity after surgical removal of the tumor and another tissue characteristic that is relevant for the therapy is determined in the reference coordinate system in a spatially resolved manner; and,
wherein at least an outer part of the x-ray applicator is made taking into account at least one of the density of the tumor cells determined in a spatially resolved manner and the other tissue characteristic that is determined.
